(19) United States
(12) Patent Application Publication
(10) Pub. No.: US 2005/0095271 A1
(43) Pub. Date: May 5, 2005

(54) Electrolyte Energy Gel

(75) Inventor: Michael S. Mathewson, San Juan Capistrano, CA (US)

Correspondence Address: Michael S. Mathewson;Crank Sports, Inc., 27136 Paseo Espada, Suite 101, San Juan Capistrano, CA, 92675, US

(73) Assignee: CRANK SPORTS, INC., San Juan Capistrano, CA (US)

(21) Appl. No.: 10/904,109

(22) Filed: Oct. 23, 2004

Related U.S. Application Data
(60) Provisional application No. 60/514,195, filed on Oct. 23, 2003.

(51) Int. Cl. A61K 47/00

(52) U.S. Cl. 424/439

(57) ABSTRACT

A nutritional composition in gel form containing carbohydrates, electrolytes, anti-oxidants, vitamins and amino acids for increasing available energy during exercise, for replenishing electrolytes that are depleted during exercise, for promoting fluid retention during exercise, for preventing hyponatremia during exercise and for facilitating intestinal reabsorption of fluids during exercise. The primary electrolyte ion is sodium (Na.sup.+) compounds is in an amount such that the sodium content of the gel is in a ratio in the range of 0.4 to 1.2 parts of the sodium to 100.0 parts of the carbohydrates.

### BACKGROUND OF INVENTION

Extensive research has been performed over the past 25 years on the effects of sports nutritional products consumed immediately prior to and during athletic performance. Additional studies have examined water consumption, hydration and electrolyte balance during athletic performance. As a general reference, much of this research has been performed or reported on by the American College of Sports Medicine. The following findings are relevant to the present invention:
(1) Consuming 0.7 g carbohydrate/kg body weight per hour (approximately 30 to 60 g per hour) has been shown unequivocally to extend athletic performance during events lasting 1 hour or more;
(2) Carbohydrate consumption in amounts typically provided in sports drinks and gels improves athletic performance in events lasting less than 1 hour, especially in athletes who exercise in the morning after an overnight fast when liver glycogen levels are low;
(3) Solutions made with maltodextrin and/or other complex carbohydrates have a lower osmolality compared to solutions made with similar amounts of sucrose and/or other simple sugars. Thus, solutions made with maltodextrin and/or other complex carbohydrates contain more energy in an isotonic state compared to solutions made with similar amounts of sucrose and/or other simple sugars;
(4) Solutions made with multiple forms of carbohydrates can deliver more energy during exercise (depending on the sources) than a solutions made with a single carbohydrate source due to the body's use of multiple energy receptors;
(5) Athletes dissipate the metabolic heat produced during physical activity by radiation, conduction, convection, and by vaporization of water. In hot, dry environments, evaporation accounts for more than 80% of metabolic heat loss. Sweat rates will vary depending on variables such as body size, exercise intensity, ambient temperature, humidity, and accumulation, but can exceed 1.8 kg (approximately 1,800 mL) per hour. In addition to water, sweat also contains substantial amounts of sodium (an average of approximately 1 g·L⁻¹) and modest amounts of potassium;
(6) Hypertonic solutions consumed during exercise may cause a net movement of fluid into the intestinal lumen because of their high osmolality. This can result is an effective loss of water from the vascular compartment and can exacerbate the effects of dehydration. Thus, it is important to bring the gel concentration down to an isotonic level of concentration (equivalent to body fluid concentration) soon after it is ingested. Once the gel/water solution reaches an isotonic state it can be rapidly absorbed by the body;
(7) Euhydration can be accomplished during exercise only if the rate of fluid ingestion and absorption equals the rate of loss through sweating (and, in events of longer duration, urination);
(8) Athletes should attempt to maintain fluid balance, as even partial dehydration can compromise performance;
(9) Dehydration also increases the risk of potentially life-threatening heat injury such as heat stroke;
(10) Fluid absorption is limited by the gastric emptying rate;
(11) Gastric emptying is reduced when hypertonic fluids are consumed and increased when isotonic or hypotonic fluids are consumed;
(12) Including sodium and potassium in an ingested solution replenishes sodium and potassium lost through sweat, thus helping to maintain electrolyte balance;
(13) Including sodium in an ingested solution in amounts between 0.5 and 0.7 g·L⁻¹ is recommended during exercise lasting longer than 1 hour because it is advantageous in promoting fluid retention and facilitating intestinal reabsorption of fluids, thus reducing the likelihood of dehydration; and
(14) Including sodium in an ingested solution in amounts between 0.5 and 0.7 g·L⁻¹ is recommended during exercise lasting longer than 1 hour because it may prevent hyponatremia in certain individuals who drink excessive quantities of fluid.

Taking all of these facts into consideration, it can be seen that a nutritional composition designed to address hydration, energy replacement, and electrolyte balance has the potential to significantly enhance athletic performance. Furthermore, many of the nutritional compositions available to athletes today are not suitable for use immediately before or during athletic performance for one or more reasons:
Products that contain fats, proteins and/or fiber (such as energy bars) that require digestion can cause discomfort. In addition, the stomach's blood requirements to facilitate the digestive process may reduce the available blood flow to working muscles that is required to facilitate the oxygenation process.
Products that contain significant water weight (such as sports drinks) may be impractical to transport during athletic performance due to bulk and/or weight.

Energy gels were introduced to the market in the early 1990's and were designed to be a quick and convenient source of energy immediately before and during athletic performance. Most of the gels also contain some combination of amino acids, vitamins and/or stimulants. None of the products however were designed to provide complete and/or balanced electrolyte replacement. Instead, athletes have had to depend on an electrolyte sports drink or an electrolyte supplement to addition to their energy gel to fulfill their electrolyte replacement requirements, The manufacturer of one of the leading energy gels (GU) also produces a sports drink that they recommend consuming with the gel.

One of the great advantages of energy gels is that they pass radidly through the stomach to the small intestine where they are absorbed through osmosis. When taken properly, the energy benefits from the gel can begin to assist the athlete very rapidly. However, for the desired osmosis process to occur, the ingested solution needs to be at an equal or lower osmolality than athlete's cellular fluids. As packaged, the osmolality of energy gels can be 10 times that of the cellular fluids, thus requiring significant amounts of water (or other low osmolality fluids) to properly dilute the gel. Optimum results can be achieved when the athlete maintains a balance between gel and fluid intake such that the combined ingested solution is isotonic (equal in concentration to the cellular fluids). Isotonic solutions are rapidly absorbed by the body and can provide significant amounts of supplemental energy during athletic performance. Solutions that are too dilute (hypotonic) will hydrate the athlete, but provide less energy benefits. Solutions that are too concentrated (hypertonic) can actually cause dehydration as the body draws cellular fluids back into the small intestine in order to bring the ingested solution down to the concentration level of the cellular fluids (isotonic). While this is happening, the energy benefits of the gel are delayed and the athlete may experience intestinal discomfort.

Sports drinks in general are designed to be stand alone products when used during athletic performance, as can be determined by examining their osmolality levels. Most sports drinks are either isotonic or slightly hypertonic. The problem with using a sports drink with an energy gel is that it is impossible for an isotonic or hypertonic solution to dilute an extremely hypertonic gel down to an isotonic osmolality level. This can only be done practically with water or some other very low osmolality fluid.

The purpose of the present invention is to design a product that provides all of the energy benefits of an energy gel, along with complete and balanced electrolyte replacement and hydration when taken with water as recommended.

Competitive Products

Traditional energy gels are broadly defined as nutritional compositions in gel form that provide energy from carbohydrates. Some traditional energy gels contain complex carbohydrates, some contain simple sugars and others contain carbohydrate blends. An effort was made to identify all traditional energy gels regardless of the carbohydrate source(s) used. Thirteen traditional energy gels have been detailed in Table 2.

Protein-based gels were not included in the competitive analysis because the present invention is for a nutritional composition that provides effectively all of it's calories (energy) from carbohydrate sources.

Table 1 provides information on the present invention (electrolyte energy gel) including the preferred formulation as well as low sodium and high sodium variations. All variations of the present invention have a ratio of sodium to carbohydrate that is significantly higher than all of the traditional energy gels (Table 2).

TABLE 1
Sodium to carbohydrate ratios of present invention

| | | Sodium per | Ratio of |
|---|---|---|---|
| | | 25 g of | sodium to |
| Product | Flavor(s) | carbohydrate | carbohydrate |
| e-Gel (invention - | All (mountain rush, | 155 mg | .62:100 |
| preferred formulation) | vanilla strawberry | | |
| | slam, tropical blast, | | |
| | cherry bomb | | |
| e-Gel (invention - low | All | 100 mg | .40:100 |
| sodium variation) | | | |
| e-Gel (invention - | All | 300 mg | 1.20:100 |
| high sodium | | | |
| variation) | | | |

TABLE 2
Sodium to carbohydrate ratios of traditional energy gels

| | | Sodium per 25 g | Ratio of sodium to |
|---|---|---|---|
| Product | Flavor(s) | of carbohydrate | carbohydrate |
| PowerGel | All (chocolate, green apple, lemon | 41 mg | .16:100 |
| | lime, raspberry cream, strawberry | | |
| | banana, tangerine, tropical fruit, | | |
| | vanilla) | | |
| GU | vanilla bean, just plain | 40 mg | .16:100 |
| | tri berry, orange, banana blitz | 45 mg | .18:100 |
| | chocolate (flavor also contains fat) | 69 mg | .27:100 |
| Clif SHOT | All (viva vanilla, razz sorbet, mocha | 52 mg | .21:100 |
| | mocha, mmm . . . chocolate, sonic | | |
| | strawberry) | | |
| Carb BOOM | All (apple cinnamon, banana peach, | 46 mg | .18:100 |
| | strawberry kiwi, vanilla orange, | | |
| | chocolate cherry) | | |
| Hammer Gel | plain | 29 mg | .12:100 |
| | banana, orange | 25 mg | .10:100 |
| | chocolate, espresso | 24 mg | .10:100 |
| | raspberry | 24 mg | .08:100 |
| Honey Stinger | All (gold, mint, ginsting, chocolate, | 43 mg | .17:100 |
| | banana) | | |
| Lava Gel | All (kona mocha, paradise peach, | 30 mg | .12:100 |
| | tropical mango, lemon wave) | | |
| HIGH5 | All (orange) | 18 mg | .07:100 |
| GoJus | All | 19 mg | .08:100 |
| Fireball Gel | All | 0 mg | .00:100 |
| Speed Gel | apple cinnamon | 30 mg | .12:100 |
| | raspberry | 21 mg | .08:100 |
| | vanilla | 29 mg | .12:100 |
| | espresso | 23 mg | .09:100 |
| EnerGel | All (orange) | 46 mg | .19:100 |
| Gulp ‘N Go | All (vanilla, orange) | 32 mg | .13:100 |
| Table 2 Notes | | | |
| (1) Lava Gel packaging indicates that it contains 75 mg of sodium per 25 g of carbohydrate, however, the product actually contains 75 mg of sodium chloride according to information available on the company's web site. Each 1.0 mg of sodium chloride contains approximately 0.4 mg of sodium, therefore the actual sodium | | | |
| # content is approximately 30 mg of sodium per 25 g of carbohydrate (see www.lavagel.com/nutrition.htm) | | | |

(2) HIGH5 is produced in the U.K. with no current distribution in the United States.
(3) GoJus is from France and the current state of distribution unknown (unable to contact company, www.gojus.com offline).
(4) Information from Fireball Gel (Canada) does not include sodium details, but the ingredient list does not include any significant sources of sodium (see www.fireballgel.com).
(5) Speed Gel and EnerGel were introduced to the market after the present invention.

### DETAILED DESCRIPTION OF INVENTION

The present invention is a nutritional composition in gel form for increasing available energy during exercise, for replenishing electrolytes that are depleted during exercise, for promoting fluid retention during exercise, for preventing hyponatremia during exercise and for facilitating intestinal reabsorption of fluids during exercise.

The ability of the composition to achieve the desired results (above) is dependent on the amount and types of carbohydrates used, the amount of sodium (and possible other electrolytes) used, and the ratio of sodium (and possibly other electrolytes) to the carbohydrates.

There are several “tradition” energy gels on the market today (see Table 2), that are primarily designed to provide quick and convenient energy before, during or immediately following athletic activity. The present invention differs from traditional energy gels in that it includes electrolytes in significantly higher levels to provide balanced electrolyte replacement in addition to the benefits of traditional energy gels. Traditional energy gels contain incidental levels of electrolytes and they are clearly not designed to meet the electrolyte replacement requirements of athletes. The specific electrolyte levels in the present invention are not arbitrarily higher than traditional energy gels, instead they have been specifically designed to work in conjunction with water and the carbohydrates in the gel to provide optimum levels of electrolytes at the point of absorption. For this reason, an upper and lower range on the ratio of sodium (the primary electrolyte in the invention) to carbohydrate has been defined. The low end of the range is more than double that of the leading energy gels, so the difference is significant and unique. The upper end of the range has also been identified to limit the scoop of the present invention and not restrict others that wish to develop highly concentrated electrolyte products for purposes not intended by the present invention.

The precise amount of electrolytes included in the formulation variations is a function of the carbohydrate sources used as well as specific electrolyte related benefit goals. The sodium to carbohydrate ratio range was determined by considering the possible carbohydrate types, combinations and amounts, along with the desired performance goals. Since the ingested solution is absorbed after it is diluted down to an isotonic osmolality level, the amount of water that is absorbed with the gel can be determined based on the composition of the carbohydrates in the gel. For example, maltodextrin and other long chain carbohydrates raise the osmolality of the gel slower than sucrose and other short chain carbohydrates. The design criteria for the preferred formulation (see table 3) is to provide a minium of 500 mg sodium and 200 mg potassium per liter of absorbed fluids.

In all of the considered variations the sodium compound(s) are included in levels such that the sodium is in a ratio in the range of 0.4 to 1.2 parts of the sodium to 100.0 parts of the carbohydrates by weight. This allows the gel when taken with water to provide the athlete with balanced electrolyte replacement. This compares to traditional energy gels that typically have sodium to carbohydrate ratios below 0.2:100. As shown in Table 2, there are no products that fall within the sodium to carbohydrate ration range of the present invention.

The inclusion of electrolytes in the above stated range gives the present invention the following advantages over traditional energy gels:
Superior electrolyte replacement benefits (electrolytes are lost through sweat)
Superior fluid retention benefits (proper hydration is critical to most athletes)
Superior hyponatremia prevention benefits
Superior intestinal reabsorption of fluids during exercise

Sports nutrition products are available in a wide variety of forms including drinks, bars, powders, tablets and gels. The present invention is for a nutritional composition in gel form only. The unique qualities of the present invention in gel form provides several athletic benefits including:
Extremely concentrated form of energy and electrolytes
Easy to consume during athletic activity
Quickly absorbed by the athlete during athletic activity
Light weight and easy to transport during athletic activity
When packaged in laminated poly/foil film, easy storage requirements and long shelf life.
Pleasant taste
Relatively low cost

Several variations of the composition have been developed and tested, and currently four formulation variations (four different flavors) are being commercially manufactured and marketed under the brand name e-Gel™. All of the commercially manufactured variations have nearly identical formulations. For the purposes of this patent application we have included the specific (preferred) formulation of one flavor (marketed under the name Tropical Blast) in Table 3 below.

TABLE 3
Preferred Formulation

| Tropical Blast e-Gel Ingredient | Percentage by Weight |
|---|---|
| Maltodextrin | 60.5% |
| Water | 26.7% |
| Fructose | 9.0% |
| Citric Acid | 1.3% |
| Sodium Citrate | 1.3% |
| Potassium Citrate | 0.4% |
| Natural and/or artificial flavors | 0.3% |
| Leucine | 0.087% |
| Valine | 0.067% |
| Isoleucine | 0.034% |
| Histidine | 0.034% |
| Ascorbic Acid (Vitamin C) | 0.134% |
| Vitamin E Acetate | 0.027% |
| Sodium Benzoate | 0.05% |
| Potassium Sorbate | 0.05% |
| Pyridoxine Hydrochloride (Vitamin B6) | 0.00044% |
| Natural and/or artificial colors | 0.001% |

The currently preferred carbohydrate sources are maltodextrin and fructose, but other carbohydrates may be used in addition to or in place of these, including but not limited to: sucrose, glucose, brown rice syrup, honey, dextrose, maltose, corn syrup, high fructose corn syrup, molasses, maple syrup, beet sugar, cane sugar and sucanat. For all currently tested variations, the included carbohydrates are in the range of 60% to 97% by weight of the composition.

Water is the preferred liquid used in the manufacturing process to turn the dry ingredients into a gel form. Fruit juice may be used in addition to, or in place of the water. Also, the amount of water (liquid) required is dependent upon the other ingredients used in the composition. For example, if a syrup form of carbohydrate is used in addition to, or in place of, the maltodextrin and fructose, the water (liquid) requirements may be reduced or eliminated. The amount of water (liquid) used will also vary depending on the desired viscosity of the composition.

Sodium Citrate and Potassium Citrate are used as the primary sources of sodium and potassium (electrolytes) in the preferred composition, however, alternate forms of sodium and potassium compounds may be substituted. Other electrolytes, including but not limited to magnesium may also be included in the composition.

Flavors from both natural and artificial sources are currently used, however, a possible variation on the formulation would contain no added flavors. The amount of each flavor ingredient is dependent on the desired taste of the composition. The flavors may included, but are not limited to, apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

Branched-chain amino acids including leucine, valine, isoleucine and histidine are included in the preferred formulation. Alternate formulations may contain a sub-set of these ingredients, additional amino acids or none at all.

The amounts of vitamins E, C and B6 may vary from the preferred composition, or eliminated. In addition, other vitamins may be added.

Sodium benzoate and potassium sorbate have been included in the preferred formulation to help preserve freshness. These ingredients may be altered, eliminated and/or substituted in alternate compositions.

Colors included in the preferred composition purely for marketing and perceived taste reasons. These ingredients may be altered, eliminated and/or substituted in alternate compositions.

Manufacturing Process

The preferred method of commercial manufacturing is in a steam jacketed kettle, however, there are a number of alternate manufacturing processes that could be used with similar results. The preferred manufacturing steps are as follows (for the preferred formulation noted above):
(1) Add water to mixing tank
(2) Turn on agitator
(3) Add sodium benzoate and potassium sorbate, mix.
(4) Add vitamins and amino acids, mix.
(5) Add sodium citrate, potassium citrate, mix.
(6) Add citric acid, mix
(7) Add color, mix
(8) Add maltodextrin, mix
(9) Add fructose, mix
(10) Add flavor, mix
(11) Heat to 170 to 180 degrees Fahrenheit
(12) Package as desired

The composition is currently being packaged in a laminated poly-foil pouch. There are several alternate packaging alternatives that are suitable for holding and storing the composition including but not limited to: bottles, cans, sealed cups and pouches made of a variety of film materials.

Use and Benefits of Invention

The composition is designed to be consumed before, during or immediately following athletic activity. In order for the desired benefits to be achieved, it is important that the athlete consume water following the consumption of the composition. Every ounce of water consumed has the ability to dilute 4 grams of the gel composition down to an isotonic level of osmolality. Thus, for a 55 g serving (current size of a single pack) of the preferred formulation, it is recommended that the athlete consume approximately 14 oz of water before consuming additional servings of the composition or other sports nutrition products. By maintaining the proper ratio of gel to water, the resulting isotonic solution can be rapidly absorbed during athletic activity, thus providing hydration benefits (from the water) along with energy and electrolyte replacement benefits (from the composition). If insufficient water is consumed, the result is a hypertonic solution that may actually have a dehydrating effect on the athlete, as well as delayed energy and electrolyte benefits. If too much water is consumed, the result is a hypotonic solution that will hydrate the athlete, but with reduced energy and electrolyte replacement benefits. The recommended amount of water is dependent on the serving size, and the type and amount of carbohydrates used in the composition. Heat, humidity, intensity level, fitness level and other factors may also be considered in determining appropriate amounts of water to consume with each serving of the composition.

The amount of water required to create an isotonic solution from the composition is also important in determining the amount of electrolytes (sodium and possibly potassium, magnesium, etc.) that are to be included in the formulation as discussed above. In the preferred formulation, the athlete is receiving approximately 0.5 grams (500 mg) of sodium per liter of absorbed fluid.

In addition to energy, electrolyte and hydration benefits, the present invention also provides additional athletic benefits from the included amino acids, anti-oxidants and vitamins. The preferred formulation provides all of the benefits that have been describe herein, however, it is important to note that several of the ingredients may be modified, changed, substituted or eliminated in future variations of the present invention. Therefore, the appended claims should be construed broadly and in a manner consistent with the spirit and scope of the present invention.

1. A nutritional composition in gel form for increasing available energy during exercise, for replenishing electrolytes that are depleted during exercise, for promoting fluid retention during exercise, for preventing hyponatremia during exercise and for facilitating intestinal reabsorption of fluids during exercise, comprising:
a) one or more carbohydrates in the range of 60.0% to 99.5% by weight of said gel for increasing available energy during exercise;
b) an electrolyte ion being sodium (Na.sup.+) compounds in an amount such that the sodium content of said gel is in a ratio in the range of 0.4 to 1.2 parts of the sodium to 100.0 parts of the carbohydrates for replenishing electrolytes that are depleted during exercise, for promoting fluid retention during exercise, for preventing hyponatremia during exercise and for facilitating intestinal reabsorption of fluids during exercise;
c) water (or water activity) in the range of 5.0% to 35.0% by weight of said gel to act as a liquid carrier for dry ingredients, to create a gel with a desirable viscosity, to partially replenish fluids lost during exercise and to reduce the amount of intestinal fluids and/or consumed water necessary to facilitate absorption of the nutritional composition.

2. A nutritional composition in accordance with claim 1, wherein 97.0% to 100.0% of the calories (energy) are derived from carbohydrate sources and less 3.0% of the calories are derived from protein and/or fat sources.

3. A nutritional composition in accordance with claim 1, further including a second electrolyte ion being potassium (K.sup.+) compounds in an amount such that the potassium content of said gel is in a ratio in the range of 0.05 to 2.0 parts of the potassium to 100.0 parts of the carbohydrates for replenishing electrolytes lost during exercise and for facilitating intestinal reabsorption of fluids.

4. A nutritional composition in accordance with claim 1, further including a vitamin compound being vitamin C in the range of 0.05% to 0.25% by weight of said composition for use as an anti-oxidant for preventing free radical formation during exercise and for protecting muscle cell integrity during exercise.

5. A nutritional composition in accordance with claim 1, further including a vitamin compound being vitamin E in the range of 0.01% to 0.10% by weight of said composition for use as an anti-oxidant for preventing free radical formation during exercise and for protecting muscle cell integrity during exercise.

6. A nutritional composition in accordance with claim 1, further including a vitamin compound being vitamin B6 in the range of 0.0001% to 0.0050% by weight of said composition to assist in the energy conversion process.

7. A nutritional composition in accordance with claim 1, further including one or more branched-chain amino acids consisting of leucine, valine, isoleucine, histidine and alanine amino acids in the range of 0.10% to 2.0% by weight of said composition for providing energy during exercise and for repairing muscle cell damage after exercise.

8. A nutritional composition in accordance with claim 1, wherein said carbohydrates are selected from the group consisting of aldohexoses, disaccharides, polysaccharides and ketohexoses such carbohydrates being glucose, glucose polymers, dextrose, maltose, maltodextrins, maltotriose, lactose, galactose, sucrose, corn syrup, high fructose corn syrup, honey, maple syrup, molasses, brown rice syrup, beet sugar, cane sugar, sucanat, arabinose, ribose, xylose, fructose, levulose, psicose, sorbose, tagatose and sorbitol.

9. A nutritional composition in accordance with claim 1, wherein said sodium (Na.sup.+) compounds are selected from the group consisting of sodium citrate, sodium chloride, sodium acetate, acidic sodium citrate, acidic sodium phosphate, sodium amino salicylate, sodium bicarbonate, sodium bromide, sodium lactate, sodium phosphate, sodium salicylate, anhydrous sodium sulphate, sodium sulphate, sodium tartrate, sodium benzoate, sodium selenite, sodium molybdate and sea salt.

10. A nutritional composition in accordance with claim 1, further including one or more flavor components from natural and/or artificial extracts including but not limited to, apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

11. A nutritional composition in accordance with claim 1, further including one or more natural and/or artificial dyes for imparting a characteristic color.

12. A nutritional composition in gel form for increasing available energy during exercise, for replenishing electrolytes that are depleted during exercise, for promoting fluid retention during exercise, for preventing hyponatremia during exercise and for facilitating intestinal reabsorption of fluids during exercise, comprising:
a) one or more carbohydrates in the range of 60.0% to 99.5% by weight of said gel for increasing available energy during exercise;
b) an electrolyte ion being sodium (Na.sup.+) compounds in an amount such that the sodium content of said gel is in a ratio in the range of 1.0 mg/calorie to 3.0 mg/calorie for replenishing electrolytes that are depleted during exercise, for promoting fluid retention during exercise, for preventing hyponatremia during exercise and for facilitating intestinal reabsorption of fluids during exercise;
c) water (or water activity) in the range of 5.0% to 35.0% by weight of said gel to act as a liquid carrier for dry ingredients, to create a gel with a desirable viscosity, to partially replenish fluids lost during exercise and to reduce the amount of intestinal fluids and/or consumed water necessary to facilitate absorption of the nutritional composition.

13. A nutritional composition in accordance with claim 12, wherein 97.0% to 100.0% of the calories (energy) are derived from carbohydrate sources and less 3.0% of the calories are derived from protein and/or fat sources.

14. A nutritional composition in accordance with claim 12, further including a second electrolyte ion being potassium (K.sup.+) compounds in an amount such that the potassium content of said gel is in a ratio in the range of 0.05 to 2.0 parts of the potassium to 100.0 parts of the carbohydrates for replenishing electrolytes lost during exercise and for facilitating intestinal reabsorption of fluids.

15. A nutritional composition in accordance with claim 12, further including a vitamin compound being vitamin C in the range of 0.05% to 0.25% by weight of said composition for use as an anti-oxidant for preventing free radical formation during exercise and for protecting muscle cell integrity during exercise.

16. A nutritional composition in accordance with claim 12, further including a vitamin compound being vitamin E in the range of 0.01% to 0.10% by weight of said composition for use as an anti-oxidant for preventing free radical formation during exercise and for protecting muscle cell integrity during exercise.

17. A nutritional composition in accordance with claim 12, further including a vitamin compound being vitamin B6 in the range of 0.0001% to 0.0050% by weight of said composition to assist in the energy conversion process.

18. A nutritional composition in accordance with claim 12, further including one or more branched-chain amino acids consisting of leucine, valine, isoleucine, histidine and alanine amino acids in the range of 0.10% to 2.0% by weight of said composition for providing energy during exercise and for repairing muscle cell damage after exercise.

19. A nutritional composition in accordance with claim 12, wherein said carbohydrates are selected from the group consisting of aldohexoses, disaccharides, polysaccharides and ketohexoses such carbohydrates being glucose, glucose polymers, dextrose, maltose, maltodextrins, maltotriose, lactose, galactose, sucrose, corn syrup, high fructose corn syrup, honey, maple syrup, molasses, brown rice syrup, beet sugar, cane sugar, sucanat, arabinose, ribose, xylose, fructose, levulose, psicose, sorbose, tagatose and sorbitol.

20. A nutritional composition in accordance with claim 12, wherein said sodium (Na.sup.+) compounds are selected from the group consisting of sodium citrate, sodium chloride, sodium acetate, acidic sodium citrate, acidic sodium phosphate, sodium amino salicylate, sodium bicarbonate, sodium bromide, sodium lactate, sodium phosphate, sodium salicylate, anhydrous sodium sulphate, sodium sulphate, sodium tartrate, sodium benzoate, sodium selenite, sodium molybdate and sea salt.

21. A nutritional composition in accordance with claim 12, further including one or more flavor components from natural and/or artificial extracts including but not limited to, apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, watermelon and equivalents thereof.

22. A nutritional composition in accordance with claim 12, further including one or more natural and/or artificial dyes for imparting a characteristic color.
